(19) United States
(12) Patent Application Publication
(10) Pub. No.: US 2023/0348687 A1
(43) Pub. Date: Nov. 2, 2023

(54) System and Method For The Biodegradation of Bio-Based Polymers In Soils and Landfills

(71) Applicant: Kimberly-Clark Worldwide, Inc., Neenah, WI (US)

(72) Inventor: Stephen Quirk, Alpharetta, GA (US)

(21) Appl. No.: 18/006,048

(22) PCT Filed: Jul. 7, 2021
(86) PCT No.: PCT/US2021/040649

§ 371 Date: Jan. 19, 2023

Related U.S. Application Data
(60) Provisional application No. 63/059,547, filed on Jul. 31, 2020.

(51) Int. Cl. C08J 11/10; C08G 63/06; C12N 11/087

(52) U.S. Cl. CPC C08J 11/105; C08G 63/06; C12N 11/087; C08J 2367/04; C12R 2001/065

(57) ABSTRACT

A process and system are disclosed for increasing the rate of degradation of biopolymers in a solid waste depository, such as a landfill. A microorganism product, which can be an encapsulated product, is combined with the waste materials. The product contains one or more microorganisms that are designed to secrete an enzyme that degrades the biopolymer, which can be a polyhydroxybutyrate polymer. The microorganism can naturally secrete the enzyme or can be genetically modified to secrete the enzyme. The microorganisms or bacteria incorporated into the product are particularly selected in order to thrive in a particular environmental condition where the solid waste is located.

### CROSS REFERENCE TO RELATED APPLICATIONS

This application claims priority to U.S. Provisional Patent Application No. 63/059,547, having a filing date of Jul. 31, 2020, which is incorporated herein by reference.

### BACKGROUND

Global production of petroleum-based plastics continues to increase every year. In recent years, for instance, over 300,000,000 metric tons of petroleum-based polymers have been produced each year. A significant portion of the above polymers are used to produce single-use products, such as plastic drinking bottles, straws, packaging, and incontinence products. Most of these plastic products are discarded and do not enter the recycle stream.

It has long been hoped that biodegradable polymers produced from renewable resources (hereinafter termed “biopolymers”) would hold great promise in reducing the global accumulation of petroleum-based plastics in the environment. For example, significant research has been done on biologically derived polymers and on polymers that biodegrade in suitable environments. One such class of biopolymers are the polyhydroxyalkanoates. Specifically, polyhydroxybutyrate (PHB) shows promise in that the polymer is derived from natural sources, can be biodegraded by several mechanisms, and is biocompatible with human tissues. Of particular advantage, polyhydroxybutyrate has thermoplastic properties that are very similar to some petroleum-based polymers.

Polyhydroxyalkanoates are synthesized using a variety of bacterial genera, including Azotobacter, Ralstonia, Burkholderia, Protomonas, Bacillus, and Schlegelella. The polyhydroxyalkanoate serves as an energy sink for these organisms. Production of polyhydroxyalkanoate polymers by the above microorganisms involves a three-step enzymatic mechanism that begins with acetyl coenzyme A. The final step of the pathway involves the polymerization of hydroxyalkanoic acid monomers into a polyhydroxyalkanoate polymer via a polyhydroxyalkanoate polymerase. Bio-synthesized polyhydroxyalkanoates accumulate in the bacterial cell as large molecular weight granules and can account for from about 60% to about 90% of the cellular dry mass.

Replacing petroleum-based polymers with biopolymers, such as polyhydroxyalkanoate polymers, would make significant advances in waste disposal processes. Even though biopolymers are capable of biodegrading significantly faster than petroleum-based polymers, biopolymers can still remain in landfills or in the soil once discarded for significant periods of time. Thus, a need exists for a system and process for accelerating the decomposition of biopolymers, such as polyhydroxyalkanoates, once they enter the solid waste stream.

### SUMMARY

In general, the present disclosure is directed to a process and system for accelerating the decomposition of polyhydroxyalkanoate polymers, particularly after the polymers have been used and discarded. Currently, a significant portion of incontinence products and personal care garments, such as diapers, child training pants, disposable swim pants, feminine hygiene products, and adult incontinence products are made from petroleum-based polymers. Significant efforts are currently underway to replace the petroleum-based polymers with biopolymers, such as polyhydroxyalkanoates that will more quickly biodegrade once buried in the soil or in a landfill. The present disclosure is directed to a process and system that more rapidly accelerates the degradation of biopolymers, once the polymers enter the solid waste stream.

In one aspect, the present disclosure is directed to a process for decreasing polymer waste material. The process includes adding to a waste material depository an encapsulated microorganism product. The waste material depository contains waste products comprising a polyhydroxyalkanoate polymer. The encapsulated microorganism product comprises at least one type of microorganism contained in a dehydrated polymer carrier. The encapsulated microorganism product is contacted with moisture. The dehydrated polymer carrier absorbs the moisture and rehydrates thereby releasing the at least one type of microorganism. The at least one type of microorganism secretes an enzyme that degrades the polyhydroxyalkanoate polymer and increases the rate at which the polyhydroxyalkanoate polymer breaks down in the waste material depository.

The enzyme secreted by the microorganism can be a polyhydroxyalkanoate depolymerase, such as poly[R-3-hydroxybutyrate] depolymerase. The microorganism contained in the encapsulated microorganism product can be a naturally occurring microorganism that naturally encodes the enzyme that is secreted. Alternatively, the microorganism can be genetically modified in order to secrete the enzyme. In one embodiment, for instance, the microorganism is a bacterium selected from Azotobacter, Ralstonia, Burkholderia, Protomonas, Bacillus, Schlegelella, or mixtures thereof. For example, in one embodiment, the at least one type of microorganism comprises Azotobacter vinelandii bacterium. Alternatively, the at least one microorganism can be a genetically modified bacterium from Escherichia coli. When using a genetically modified microorganism, in one aspect, the depolymerase gene can be inserted into a chromosome of the microorganism by transduction or linear recombination. Alternatively, an extra chromosomal vector can be inserted into the microorganism.

The amount of the encapsulated microorganism product added to the waste material depository can depend upon a weight ratio between the encapsulated microorganism product and the amount of solid waste present. Alternatively, the amount of the encapsulated microorganism product can be based on a weight ratio between the product and the amount of polyhydroxyalkanoate polymers present in the waste material depository. In still another aspect, the amount of the encapsulated microorganism product added to the waste material depository can be based on the number of viable microorganisms that produce the desired enzyme in relation to the amount of polyhydroxyalkanoate polymers present or based upon the overall weight of the waste material depository.

In one aspect, the waste material depository contains used incontinence products made from a polyhydroxyalkanoate polymer. The incontinence products, for instance, can include a liquid permeable liner, an outer cover, and an absorbent structure positioned between the liquid permeable liner and the outer cover. The liquid permeable liner and/or the outer cover can be made from a polyhydroxyalkanoate. The encapsulated microorganism product can be added to the waste material depository in order to accelerate the degradation of the used incontinence products. In fact, in one embodiment, the amount of the encapsulated microorganism product can be added to the waste material depository based upon the amount of used incontinence products contained within the waste material depository.

In one aspect, the waste material depository can contain soil and can comprise a landfill. The waste material depository can be located in a particular environment that provides the moisture needed to contact with the encapsulated microorganism product. In one aspect, the at least one type of microorganism selected for encapsulation within the microorganism product can be particularly selected based upon environmental variables of where the waste material depository is located. These environmental variables can include temperature, salinity, the concentration of oxygen present, and combinations thereof.

As described above, the encapsulated microorganism product includes at least one type of microorganism contained within a dehydrated polymer carrier. The dehydrated polymer carrier, in one embodiment, can be a crosslinked polyacrylate. In other applications, however, the dehydrated polymer carrier can comprise a starch-based polymer, a cellulose-based polymer, agarose, or a crosslinked polyvinyl alcohol polymer. In one aspect, one or more microorganisms can be loaded into the polymer carrier while the polymer carrier is in a gel-like state. The polymer carrier can then be dehydrated to produce a solid product. The solid product can then be cut, chopped, or ground to a desired size. The encapsulated microorganism product, for instance, can comprise flakes, particles, granules, fibers, or the like.

In another aspect, the present disclosure is directed to a process for decreasing polymer waste material comprising adding to a waste material depository a genetically modified microorganism. The waste material depository containing waste products comprising a polyhydroxyalkanoate polymer. The genetically modified microorganism having been modified to secrete an enzyme that degrades the polyhydroxyalkanoate polymer and increase a rate at which the polyhydroxyalkanoate polymer breaks down in the waste material depository.

Other features and aspects of the present disclosure are discussed in greater detail below.

### BRIEF DESCRIPTION OF THE DRAWINGS

A full and enabling disclosure of the present disclosure is set forth more particularly in the remainder of the specification, including reference to the accompanying figures, in which:

FIG. 1 is a cross-sectional view of the test setup for the examples described below;

FIG. 2 is a graphical representation of some of the results obtained in the examples below;

and

FIG. 3 is a graphical representation of some of the results obtained in the examples below.

Repeat use of reference characters in the present specification and drawings is intended to represent the same or analogous features or elements of the present invention.

### DETAILED DESCRIPTION

It is to be understood by one of ordinary skill in the art that the present discussion is a description of exemplary embodiments only and is not intended as limiting the broader aspects of the present disclosure.

Part of the sustainability equation in order to reduce and eliminate polymer waste is to replace petroleum-based polymers with polymers obtained from renewable energy sources that are also biodegradable. Significant research is currently underway to improve processes for mass producing biopolymers and to techniques and methods for improving the physical properties of the polymers. Such polymers are well suited to producing all different types of single-use products, such as drink bottles, containers, packaging, and the like. In addition, those skilled in the art have proposed in the past replacing petroleum-based polymers found in disposable incontinence products with biopolymers, such as polyhydroxyalkanoate polymers. The use of biopolymers to replace petroleum-based polymers will make significant strides in creating a sustainable economy.

Most single-use products, such as incontinence products, are buried in landfills or buried in the soil after use. Even if made from biopolymers, these single-use products will still require a significant amount of time to degrade. Consequently, in order to further improve the sustainability equation, the present disclosure is directed to a method and system for accelerating degradation of biopolymers once the biopolymers have entered the solid waste stream.

In this regard, the present disclosure is generally directed to adding to a waste material depository a microorganism population that is particularly selected to secrete an enzyme for significantly increasing the rate at which the biopolymers degrade. In one aspect, the microorganisms are encapsulated but remain viable and are released from the encapsulation once deposited in the waste material depository. The process and system of the present disclosure is particularly directed to rapidly degrading used products containing polyhydroxyalkanoate polymers using microorganisms, such as bacteria or archaea, that secrete an appropriate depolymerase enzyme.

Any suitable polyhydroxyalkanoate polymer can be degraded and decomposed according to the present disclosure. The polyhydroxyalkanoate polymer can be a homopolymer or a copolymer. Polyhydroxyalkanoates, also known as “PHAs”, are linear polyesters produced in nature by bacterial fermentation of sugar or lipids. More than 100 different monomers can be combined within this family to produce materials. One common type of polyhydroxyalkanoate polymer is Poly(3-hydroxybutyrate) (PHB).

Examples of monomer units that can be incorporated in polyhydroxyalkanoate polymers include 2-hydroxybutyrate, glycolic acid, 3-hydroxybutyrate, 3-hydroxypropionate, 3-hydroxyvalerate, 3-hydroxyhexanoate, 3-hydroxyheptanoate, 3-hydroxyoctanoate, 3-hydroxynonanoate, 3-hydroxydecanoate, 3-hydroxydodecanoate, 4-hydroxybutyrate, 4-hydroxyvalerate, 5-hydroxyvalerate, and 6-hydroxyhexanoate.

Examples of polyhydroxyalkanoate homopolymers include poly 3-hydroxyalkanoates (e.g., poly 3-hydroxypropionate (PHP), poly 3-hydroxybutyrate (PHB), poly 3-hydroxyvalerate (PHV), poly 3-hydroxyhexonoate (PHH), poly 3-hydroxyoctanoate (PHO), poly 3-hydroxydecanoate (PHD), and poly 3-hydroxy-5-phenylvalerate (PHPV)), poly 4-hydroxyalkanoates (e.g., poly 4-hydroxybutyrate (hereinafter referred to as PHB) and poly 4-hydroxyvalerate (hereinafter referred to as PHV)), or poly 5-hydroxyalkanoates (e.g., poly 5-hydroxyvalerate (hereinafter referred to as PHV)).

In certain embodiments, the PHA can be a copolymer (containing two or more different monomer units) in which the different monomers are randomly distributed in the polymer chain. Examples of PHA copolymers include poly 3-hydroxybutyrate-co-3-hydroxypropionate (hereinafter referred to as PHB3HP), poly 3-hydroxybutyrate-co-4-hydroxybutyrate (hereinafter referred to as P3HB4HB), poly 3-hydroxybutyrate-co-4-hydroxyvalerate (hereinafter referred to as PHB4HV), poly 3-hydroxybutyrate-co-3-hydroxyvalerate (hereinafter referred to as PHB3HV), poly 3-hydroxybutyrate-co-3-hydroxyhexanoate (hereinafter referred to as PHB3HH) and poly 3-hydroxybutyrate-co-5-hydroxyvalerate (hereinafter referred to as PHBSHV).

The microorganism or collection of microorganisms that are selected for use in the present disclosure can be selected not only in order to secrete a particular enzyme, but can also be selected based upon the environmental conditions in which the waste material depository exists. For example, the waste material depository can be contained in one of numerous environments that may be defined by a particular annual temperature range, salinity amounts, and the amount of oxygen contained in the soil or landfill. In accordance with the present disclosure, the particular environmental variables present at a particular location can be matched to one or more microorganisms, such as bacteria and/or archaea, best suited for the particular environment. The microorganism selected, for instance, can be a microorganism that naturally produces the desired enzyme or can be a microorganism that has been genetically modified or cloned in order to express the desired depolymerase gene.

In general, any suitable microorganism can be selected for use in the product of the present disclosure that secretes a metabolite or enzyme capable of degrading a biopolymer, particularly a polyhydroxyalkanoate polymer. For instance, the one or more microorganisms can be one or more bacteria or archaea that either expresses a native or an exogenous poly(hydroxybutyrate) depolymerase enzyme. In one particular embodiment, the enzyme can be a poly[R-3-hydroxybutyrate] depolymerase enzyme. The following reaction, for instance, illustrates the enzymatic degradation of a polyhydroxybutyrate polymer by a poly[R-3-hydroxybutyrate] depolymerase.

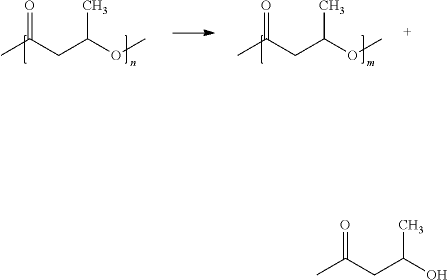

wherein m<<<n and represents small oligomers.

As stated above, in one aspect, the enzyme or metabolite that breaks down the polyhydroxyalkanoate polymer can be a naturally occurring bacteria that naturally expresses the desired enzyme. For instance, in one aspect, the microorganism incorporated into the product of the present disclosure is selected from a variety of bacterial genera including Azotobacter, Ralstonia, Burkholderia, Protomonas, Bacillus, or Schlegelella. One particular bacterium well suited for use in the product of the present disclosure, for instance, is Azotobacter vinelandii. Azotobacter vinelandii, for instance, can survive over a relatively broad temperature range, is salinity resistant, and can produce significant amounts of an enzyme for breaking down polyhydroxyalkanoate polymers.

In addition to microorganisms that naturally express the depolymerase gene, one or more genetically modified bacteria may also be selected that express an exogenous depolymerase enzyme capable of breaking down a polyhydroxyalkanoate polymer. For example, in accordance with the present disclosure, any genus of bacterium or archaean can be matched with any polyhydroxyalkanoate depolymerase enzyme that is expressed from a constitutive vector coupled with the correct signal sequence. In this aspect, any suitable gram positive or gram negative bacterium can be used to produce and secrete the enzyme, which can be a gram positive polyhydroxyalkanoate depolymerase enzyme. In this manner, the microorganism product of the present disclosure can be customized based on environmental variables, the type and amount of solid waste materials in the waste material depository, or combinations thereof. In addition, the sequence of the enzyme can be matched to the environment by selecting one of approximately 6,400 depolymerase sequences that are known (e.g. NCBI database) or with a fully or partially engineered variant. In one aspect, the selected bacteria or archaea can be transformed with a plasmid vector which harbors a constitutively expressed gene in coding a poly[R-3-hydroxybutyrate] depolymerase that contains an appropriate N-ter signal sequence. Alternatively, the bacterium or archaean of choice can have the depolymerase gene inserted into the bacterial chromosome by transduction, linear recombination, or any other suitable method instead of using an extra chromosomal vector thereby eliminating the need for an exogenous vector.

When modifying a microorganism, any suitable gram positive or gram negative bacteria may be used. For example, in one embodiment, the modified bacteria can be obtained from the genus Streptomyces. Particular examples of microorganisms from the above genus include Streptomyces thermovulgaris, Streptomyces thermoolivaceus, Streptomyces thermohygroscopicus, Streptomyces thermocarboxydovorans, or mixtures thereof.

The following are further soil phyla and representative genera that may be selected in accordance with the present disclosure and genetically modified to express a PHB depolymerase. It should be understood that the following list is exemplary only. The below microorganisms, however, are well suited to proliferating in soils. The particular microorganism can be selected based on soil content (sand versus dirt), temperature, oxygen availability, salinity, and the like.

Firmicutes: Bacillus, Lihuaxuella, and Clostridium;

Proteobacteria: Bradyrhizobium, Sphingomonas, Azotobacter, Azospirillum, Nitrobacter, Lysobacter, Stenotrophomonas, Rhizobium, Acinetobacter, Thiobacillus, Schlegelella, Janthinobacterium, Sinorhizobium, Pseudomonas, Agrobacterium, and Escherichia (e.g. Escherichia coli);

Actinobacteria: Rhodococcus, Arthobacter, Streptomyces, Conexibacter, Rhodococcus, Solirubrobacter, Micrococcus, Rubrobacter, and Actinomyces;

Bacteroidetes: Flavobacterium and Pedobacter;

Deinococcus-thermus: Deinococcus and Thermus;

Gemmatimonadetes: Gemmatimonas and Gemmatirosa;

Spirochaetes: Tumeriella and Leptospira;

Verrucomicrobia: Pedosphaera, Chthoniobacter, and Verrucomicrobia;

Chloroflexi: Thermogemmatispora and Dictyobacter; and

Armatimonadetes: Fimbriimonas

In one aspect, the microorganism product of the present disclosure can include a combination of different bacterium. For example, in one aspect, the product can contain bacteria that naturally secrete the depolymerase enzyme combined with bacteria that have been genetically modified in order to secrete the depolymerase enzyme. The genetically modified bacteria, for instance, can be used to fine tune the system based on environmental conditions and feed supply.

In one aspect, the microorganisms can be directly added to the waste material depository or added in combination with a carrier. The carrier can be a food source, can be a soil, or can be any suitable carrier.

In one embodiment, the microorganisms can be encapsulated in a carrier, such as a polymer carrier. The polymer carrier can be a material that is highly water absorbent without being water soluble. In one aspect, for instance, the polymer carrier is in the form of a gel when combined with water, can be dehydrated and converted into the form of a solid, and then capable of being rehydratable when contacted with moisture. In this manner, the one or more microorganisms can be combined with the polymer carrier in the form of a gel. Once blended together, water can then be removed in order to form a solid. The solid can be formed into any suitable shape and contacted with waste materials. In order to degrade polymers contained in the waste material, the solid material is contacted with moisture that causes the carrier polymer to rehydrate. Once rehydrated, the microorganisms can be released from the polymer gel or can secrete enzymes that are released from the polymer gel.

Various different materials can be used as the polymer carrier. For example, in one aspect, the polymer carrier can comprise a polyacrylate polymer, and particularly a crosslinked polyacrylate polymer.

For example, in one embodiment, the carrier polymer can be a salt of a polyacrylic acid that is polymerized with a crosslinking agent, such as a silane. For example, in one embodiment, the crosslinking agent can be a trimethoxysilane, such as methacryloxy-propyl-trimethoxysilane. The salt of polyacrylic acid can be a sodium salt and can be at least 40% neutralized, such as at least 50% neutralized, such as at least 60% neutralized, and less than about 90% neutralized, such as less than about 80% neutralized. The polymer can have any suitable degree of crosslinking and molecular weight so as to have a gel-like state when combined with water. For example, the polymer can have a molecular weight of greater than about 50,000 daltons, such as greater than about 100,000 daltons, such as greater than about 150,000 daltons, such as greater than about 200,000 daltons, such as greater than about 225,000 daltons, and generally less than about 500,000 daltons, such as less than about 300,000 daltons, such as less than about 275,000 daltons.

In addition to polyacrylate polymers, various other rehydratable polymers may be used as the carrier polymer in the product of the present disclosure. For example, in an alternative embodiment, the carrier polymer can be a starch, and particularly a starch copolymer. In an alternative embodiment, the carrier polymer can be a cellulose polymer, such as a methyl cellulose polymer, an ethyl cellulose polymer, or a carboxymethyl cellulose polymer. Other carrier polymers that may be used include agarose, dextran, a gelatin, a polyvinyl alcohol, and mixtures thereof. Particular examples of carrier polymers include hydrolysis products of starch-acrylonitrile graft copolymers, starch-acrylic acid graft copolymers, starch-styrenesulfonic acid graft copolymers, starch-vinylsulfonic acid graft copolymers, starch-acrylamide graft copolymers, cellulose-acrylonitrile graft copolymers, cellulose-styrenesulfonic acid graft copolymers, crosslinked carboxymethylcellulose, hyaluronic acid, agarose, crosslinked polyvinyl alcohols, sodium acrylate-vinyl alcohol copolymers, saponification products of polyacrylonitrile polymers, and combinations of two or more thereof.

As described above, the carrier polymer can be in a gel-like state when combined with the one or more microorganisms. For example, when the one or more microorganism are combined with the carrier polymer, the carrier polymer can contain water in an amount greater than about 30% by weight, such as in an amount greater than about 40% by weight, such as in an amount greater than about 50% by weight, such as in an amount greater than about 60% by weight, such as in an amount greater than about 70% by weight, such as in an amount greater than about 80% by weight, and even in amounts greater than 90% by weight depending upon the particular carrier polymer chosen. The amount of water is generally less than about 99% by weight, such as less than about 95% by weight, such as less than about 80% by weight. When using a crosslinked polyacrylate polymer, for instance, the carrier polymer can contain water in an amount from about 55% to about 85% by weight including all increments of 1% by weight therebetween.

After the carrier polymer and one or more microorganisms are combined together, the carrier polymer can be dehydrated by removing water. For instance, water can be removed from the resulting mixture in order to form a solid product. The resulting solid product can contain water in an amount less than about 20% by weight, such as in an amount less than about 10% by weight, such as in an amount less than about 8% by weight, such as in an amount less than about 6% by weight, such as in an amount less than about 4% by weight, such as in an amount less than about 2% by weight. Water is generally contained in the solid product in an amount greater than about 0.01% by weight, such as in an amount greater than about 0.5% by weight.

The resulting solid material represents an encapsulated microorganism product. The amount of one or more microorganisms contained in the product can vary depending upon the particular microorganism selected, the carrier polymer selected, and the waste materials that are to be combined with the product. In general, the amount of microorganisms per mg of encapsulated material can be in a range of 1×10 cfu to 1×10¹⁰ cfu, such as from 1×10³ cfu to 1×10⁸ cfu, such as from 1×10⁴ cfu to 1×10⁶ cfu.

In addition to one or more microorganisms, the encapsulated microorganism product of the present disclosure can also contain various other additives and components. In one particular application, for instance, a biopolymer can be added to the carrier polymer in conjunction with the one or more microorganisms. The biopolymer, for instance, can be a polyhydroxyalkanoate, such as a polyhydroxybutyrate polymer. The polyhydroxyalkanoate polymer, for instance, can serve as a food source for the microorganism within the encapsulated product and can prime the microorganism for producing the desired depolymerase enzyme. In particular, adding a polyhydroxyalkanoate polymer into the encapsulation media helps keep the metabolic functions of the microorganism focused on polyhydroxybutyrate degradation. The amount of polyhydroxyalkanoate polymer contained in the encapsulated microorganism product based on per gram of lyophilized mixture can generally be in a range of 0.1 mg to 500 mg including all increments of 0.1 mg therebetween, such as in a range of 1 mg to 200, such as in a range of 10 mg to 100 mg.

In addition to one or more microorganisms and a polyhydroxyalkanoate polymer, the encapsulated microorganism product can also contain a sugar, such as glucose or trehalose and/or a protein source, such as bovine serum albumin.

Once the carrier polymer and one or more microorganisms are combined together and dehydrated to form a solid product, the solid product can be converted into any suitable size and shape for later addition to a waste material repository, such as a landfill or soil. As used herein, soil refers to any granular ground cover including dirt, sand, humus, gravel or mixtures thereof. In one embodiment, for instance, the resulting mixture can be dehydrated and formed into a film or fibers. The fibers can then be chopped to any desired fiber size. For instance, the fibers can have an average fiber length of less than about 5 cm, such as less than about 3 cm, such as less than about 1 cm, and generally greater than about 0.01 mm, such as greater than about 0.1 mm, such as greater than about 0.5 mm.

When formed into a film, the film can be cut into the form of flakes. The flakes, for instance, can have an average particle size of less than about 10 mm, such as less than about 7 mm, such as less than about 5 mm, such as less than about 3 mm, and generally greater than about 0.1 mm, such as greater than about 0.5 mm.

In an alternative embodiment, the encapsulated microorganism product can be in the form of chunks, cubes, particles or granules. The particles or granules, for instance can be the product of a grinding process. The particles or granules, for instance, can have an average particle size of less than about 10 mm, such as less than about 5 mm, such as less than about 4 mm, such as less than about 3 mm, and generally greater than about 0.01 mm, such as greater than about 0.1 mm, such as greater than about 0.5 mm, such as greater than about 1 mm.

The encapsulated microorganism product as described above can be combined with a waste material depository for accelerating the degradation of biopolymers, particularly polyhydroxyalkanoate polymers. The encapsulated microorganism product in the form of particles, flakes, granules, or fibers, can be mixed into a waste material load or added directly to the soil or a landfill. Contact with moisture causes the polymer carrier to rehydrate. As the polymer carrier rehydrates, the polymer carrier transforms into a gel-like state and releases the microorganisms contained therein. The microorganisms secrete a depolymerase enzyme that then breaks down or degrades polyhydroxyalkanoate polymers, such as polyhydroxybutyrate polymers.

In one aspect, for instance, the microorganism population, such as an encapsulated microorganism product of the present disclosure, can be combined with a solid waste stream that contains discarded incontinence products made from a polyhydroxyalkanoate polymer. Incontinence products include, for example, diapers, training pants, swim pants, adult incontinence products, feminine hygiene products, and the like. These products typically include a water permeable liner, an outer cover, and an absorbent structure positioned between the liquid permeable liner and the outer cover. In the past, the liquid permeable liner and the outer cover were primarily made from petroleum-based polymers. In future incontinence products, however, the petroleum-based polymers will be replaced with biopolymers, such as polyhydroxybutyrate. Consequently, incontinence products may contain biopolymers in amounts greater than about 5% by weight, such as in amounts greater than about 10% by weight, such as in amounts greater than about 20% by weight, such as in amounts greater than about 30% by weight, such as in amounts greater than about 40% by weight, such as in amounts greater than about 50% by weight, such as in amounts greater than about 60% by weight, such as in amounts greater than about 70% by weight. In order to significantly increase the degradation of the discarded incontinence products, the encapsulated microorganism product of the present disclosure can be combined with these products as they enter the solid waste stream for increasing the rate of degradation.

The amount of the encapsulated microorganism product added to a waste material depository can be based on the amount of waste materials in the depository, on the amount of polyhydroxyalkanoates present in the waste material depository, or based on a ratio between the encapsulated microorganism product and the amount of soil present. When based on the amount of encapsulated microorganism product per incontinence product, the range can be from about 0.000001 g to about 10.0 g, such as from about 0.001 g to about 5.0 g, such as from about 0.1 g to about 1 g.

The present disclosure may be better understood with reference to the following example.

### Example

The following example demonstrates some of the benefits and advantages of the present disclosure.

In this example, two different bacterium were tested for their viability during encapsulation according to the present disclosure and for their ability to degrade polyhydroxybutyrate films. A bacterium was tested that naturally produces an enzyme and a bacterium was also tested that had been genetically modified to produce the enzyme. The bacteria tested that naturally produces the enzyme was Azotobacter vinelandii. The sequence of the PHBDase enzyme secreted by Azotobacter vinelandii is as follows:

>ACO79630.1 [Azotobacter vinelandii DJ]
MPRTWIFRLFVLPLLLLPAPLAWGGSLESFSLPAAGHPGSRERQYKVYL
PDVRSETPPLVMALHGCLMDNDDVLRDWGLTVAADRHGFILVAPSVSGY
DELRAPNCWGFWIDGQRHEGRGEAEDLLRIAQAVEARYRTDPARRYIAG
LSSGGAMSVVAAIAHNEYWAAAASVAGLPYGEEARAVSSAACPLSNASL
HGVERVVADMRAELDDPYPIPLLVIQNDNDCTVVAGAGRRLRDAHLKAF
GSAPFDTPAGTLAVQRRCIPVFGDDDYGCRHEIHTADGNSASRSLVETL
YYQGPLETPAENDIDRGHYWIGGEHGREGPYALRPGPSHPDILWYFFAR
HPRNGMEPDDSPRVTIEGANPLRLPLGESFVDPGARAGDARDGELAVGV
DCTVDTRRAGRYRCTYSTTNRRGKRSEAIRTVLVEDPDVPAATCATATA
SPLGHLFGGRALPGGALYGRTLASGDRTDIGSVFQGLTPVTLHEGAPGE
WFVRPPEACGW

The other microorganism studied was a transformed Escherichia coli. The Escherichia coli included the following sequence of the enzyme produced through a gram negative bacterial signal peptide replacement (in bold).

MKNITFIFFILLASPLYASLESFSLPAAGHPGSRERQYKVYLPDVRSET
PPLVMALHGCLMDNDDVLRDWGLTVAADRHGFILVAPSVSGYDELRAPN
CWGFWIDGQRHEGRGEAEDLLRIAQAVEARYRTDPARRYIAGLSSGGAM
SVVAAIAHNEYWAAAASVAGLPYGEEARAVSSAACPLSNASLHGVERVV
ADMRAELDDPYPIPLLVIQNDNDCTVVAGAGRRLRDAHLKAFGSAPFDT
PAGTLAVQRRCIPVFGDDDYGCRHEIHTADGNSASRSLVETLYYQGPLE
TPAENDIDRGHYWIGGEHGREGPYALRPGPSHPDILWYFFARHPRNGME
PDDSPRVTIEGANPLRLPLGESFVDPGARAGDARDGELAVGVDCTVDTR
RAGRYRCTYSTTNRRGKRSEAIRTVLVEDPDVPAATCATATASPLGHLF
GGRALPGGALYGRTLASGDRTDIGSVFQGLTPVTLHEGAPGEWFVRPPE
ACGW

Organism and growth conditions: The bacterium Azotobacter vinelandii was obtained from the American Type Culture Collection (ATCC BAA-1303). Cells were propagated in liquid culture or on agar plates in media A that contained (per 1 L): 5.0 g yeast extract, 2.0 g casamino acids, 8.0 g polypeptone peptone, 2.0 g NaCl, (and 15 g agar for plating). For PHB biodegradation experiments, mid-log phase Azotobacter vinelandii cultures were centrifuged for 4 mins, 10,000×g, washed in PBS, recentrifuged, and resuspended in liquid media B composed of (per 1 L): 7.0 g Na₂HPO4-7H₂O, 3.0 g KH₂PO₄, 1 g NH₄Cl, 2.0 g casamino acids, 1.0 g Na-glutamate, 2.0 g KCl, 3.0 g Na₃-citrate, 20.0 g MgSO₄-7H₂O, 2.0 g NaCl, 36.0 g FeCl₂-4H₂O, 0.5 g MnCl₂-4H₂O, 4.0 g granulated poly(3-hydroxybutyrate). All growth was at 65° C. Escherichia coli (lab stock) was propagated in standard Lauria Broth with growth at 37° C. For PHB biodegradation experiments, mid-log phase Escherichia coli cultures were centrifuged for 4 mins, 10,000×g, washed in PBS, recentrifuged, and resuspended in liquid media B (minus phosphate containing compounds). All transformed E. coli media was further supplemented with 40 μg/mL kanamycin.

PHB film preparation: PHB was solvent cast using chloroform. PHB granules were dissolved in chloroform at 70° C., with constant stirring to a final concentration of 1.0 mg/mL. Typical time for complete dissolution was 60 minutes. The solution was then poured onto a 25° C. 5.0 cm×5.0 cm glass slide (2.0 mLs typical poured volume) and air dried for 24 hours. Typically, the films were further aged for five days (air atmosphere at 25° C.) and vacuum dried for three hours prior to use. Films were not further characterized.

Encapsulated bacteria: A mid-log culture of Azotobacter vinelandii or transformed Escherichia coli in media B were lyophilized after the addition of trehalose (final concentration of 5% w/v) and bovine serum albumin (final concentration 1 mg/mL). Polyacrylic acid, sodium salt (70% neutralized), that is polymerized with methacryloxy-propyl-trimethoxysilane, was obtained from Evonik Corporation (Richmond VA, designated as polymer SR1717). It is 250 kDa oligomer and is supplied as a 32% (wt/wt) solids in a water solution. Granulated PHB, 100 mg total mass and 1 g of the lyophilized mixture was added per 5 mLs of the polyacrylate with gentle mixing and the sample was poured into the wells of a 6-well culture plate. The samples were cured in a convection oven at 40° C. for 30 minutes to drive off the water. As the water is removed, the silanol groups on the polymer chain begin to crosslink, forming a lightly cross-linked polyacrylate hydrogel. Samples were stored at 10° C. until used.

Production of an expression vector. The amino acid sequence of the Azotobacter vinelandii PHBDase (ACO79630.1) was utilized to construct a recombinant DNA expression system. The first 25 amino acids of the native sequence (the signal sequence region) were removed and replaced with a signal sequence, MKNITFIFFILLASPLYA, that functions in the gram(−) E. coli. This new sequence was reverse translated to DNA and codon optimized for expression in E. coli using the program Gene Designer from ATUM, Inc. The gene was assembled using standard PCR techniques by ATUM, Inc. and cloned into the expression vector pD1831-SR (kan^{r}, strong ribosomal binding site, PhoA promoter). The insert was verified by DNA sequencing after construction.

PHB film biodegradation assays: A 1 cm diameter plug of the encapsulated material was centered on top of the PHB film on the glass slide. That set-up was placed into a petri dish and covered with moist potting soil. At 1-day intervals, the glass slide was removed from the petri dish, gently washed with distilled water and dried under vacuum at 50° C. for an hour in order to drive off all fluid. The residual encapsulated material was removed and the PHB film/glass slide was weighed. The mass of degraded PHB was calculated as:

((film/slide mass at t=0)−slide mass)−((film/slide mass at tₙ)−slide mass)

The percent mass loss was then defined as:

[1−(film mass at tₙ/film mass at t=0)]×100

All assays were performed in triplicate and averaged.

Enzymatic reaction conditions. A turbidometric assay was employed to measure PHBDase activity under various conditions. The standard reaction (final volume=1.0 mL) contained 200 mg/L of PHB granules (that were previously stably suspended via sonication), 1 mM CaCl₂, 25 mM buffer at various pH values. The reaction was initiated after the addition of enzyme/bacteria solution and monitored at 650 nm in Applied Photophysics spectrapolarometer in absorbance mode. The reaction was gently stirred and maintained at a constant temperature. OD measurements (typically starting in the range of 2-3) were converted to percent OD remaining as a function of time or converted to percent remaining activity.

Soil stability experiments: One gram of encapsulated or unencapsulated (but lyophilized) A. vinelandii and transformed E. coli were mixed with 30 g of potting soil to which was added 5 g of distilled water in a small container. The samples were incubated at 30° C. for two months. At 0, 1, and 2-month timepoints, a 200 mg sample was taken out of the container and mixed with 500 μL of distilled water. The mixture was incubated with gentle agitation for 10 minutes at room temperature and centrifuged for 4 mins, 10,000×g. A 20 μL aliquot of the supernatant was removed and assayed for PHBDase activity using the standard assay. The amount of activity at the initial timepoint (t=0) was set to 100 percent activity.

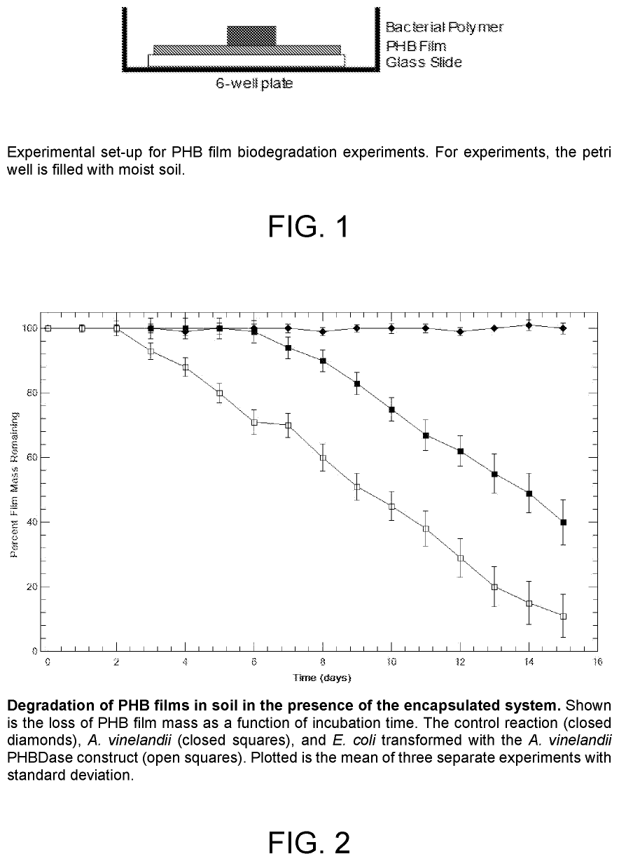

All known PHBDases for monomeric hydroxybutyrate and small oligomers of the polymer. Encapsulated A. vinelandii and encapsulated E. coli harboring the A. vinelandii PHBDase expressing construct are capable of degrading PHB films in moist soil in the set-up diagramed in FIG. 1. Degradation of PHB film occurs in a time dependent manner as is shown in FIG. 2. The control (closed diamonds) shows that the experimental technique can reproducibly measure intact PHB film mass as evidenced by the near 100 percent mass values and low standard deviation obtained throughout the experiment timecourse. A. vinelandii (closed squares) shows an approximately 6 day lag before mass reduction is seen. This presumably is a measure of the bacteria becoming metabolically active and the time to produce, secrete, and diffuse active enzyme into the soil. The reaction results in a 50% loss of PHB film mass in approximately 12.5 days. Encapsulated E. coli harboring the A. vinelandii PHBDase expressing construct (open squares) is characterized by a 3 day lag and a 9 day time to a 50% loss of PHB film mass. The faster kinetics in this system are likely due to a larger concentration of enzyme in the bacterial cell at the time of lyophilization due to constitutive plasmid expression.

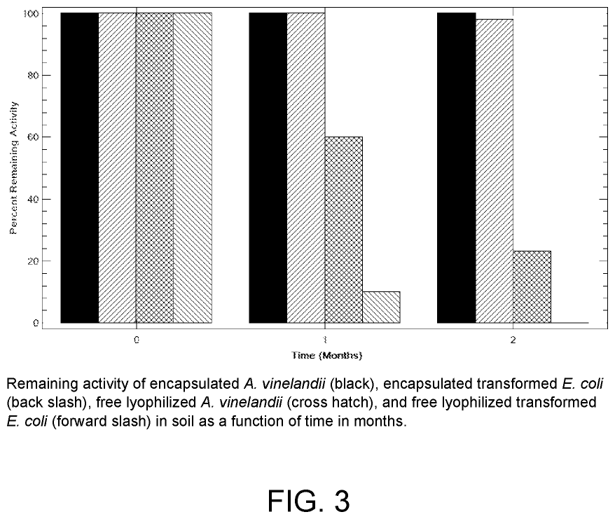

Encapsulating either A. vinelandii or transformed E. coli in the lightly cross-linked polyacrylate results in a system that is able to express active enzyme longer when in contact with moist soil than unencapsulated bacteria as is shown in FIG. 3. Encapsuled A. vinelandii retains 100 percent activity over the two months of the study versus 23 percent for free A. vinelandii. Similar results for the encapsulated transformed E. coli which retains 98% activity versus 0 percent for free transformed E. coli.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

1. A process for decreasing polymer waste material comprising:
adding to a waste material depository an encapsulated microorganism product, the waste material depository containing waste products comprising a polyhydroxyalkanoate polymer, the encapsulated microorganism product comprising at least one type of microorganism contained in a dehydrated polymer carrier; and
contacting the encapsulated microorganism product with moisture, wherein the dehydrated polymer carrier absorbs the moisture and rehydrates thereby releasing the at least one type of microorganism, the at least one type of microorganism secreting an enzyme that degrades the polyhydroxyalkanoate polymer and increases a rate at which the polyhydroxyalkanoate polymer breaks down in the waste material depository.

2. A process as defined in claim 1, wherein the enzyme secreted by the at least one type of microorganism comprises poly[R-3-hydroxybutyrate] depolymerase.

3. A process as defined in claim 1, wherein the at least one type of microorganism contained in the dehydrated polymer carrier is a naturally occurring bacterium that naturally encodes the enzyme.

4. A process as defined in any of claim 1, wherein the at least one type of microorganism comprises a genetically modified microorganism that has been genetically modified to secrete the enzyme.

5. A process as defined in claim 1, wherein the at least one type of microorganism comprises at least one type of bacterium.

6. A process as defined in claim 1, wherein the waste products contained within the waste material depository comprise incontinence products, the incontinence products comprising a polyhydroxyalkanoate polymer.

7. A process as defined in claim 6, wherein the incontinence product includes a liquid permeable liner, an outer cover, and an absorbent structure positioned in between the liquid permeable liner and the outer cover, the liquid permeable liner and the outer cover being made from a polyhydroxyalkanoate polymer.

8. A process as defined in claim 1, wherein the amount of microorganisms per mg of encapsulated microorganism product is in a range of 1×10 cfu to 1×10¹⁰ cfu.

9. A process as defined in claim 1, wherein, based on the amount of encapsulated microorganism product per incontinence product, the amount of encapsulated microorganism product added to the waste material depository is in the range of from about 0.000001 g to about 10.0 g.

10. A process as defined in claim 1, wherein the at least one type of microorganism contained within the encapsulated microorganism product comprises a bacterium selected from a bacterial genera comprising Azotobacter, Ralstonia, Burkholderia, Protomonas, Bacillus, Schlegelella, or mixtures thereof.

11. A process as defined in claim 1, wherein the at least one type of microorganism contained in the encapsulated microorganism product comprises an Azotobacter vinelandii bacterium.

12. A process as defined in claim 4, wherein the at least one type of microorganism contained within the encapsulated microorganism product comprises bacterium selected from Firmicutes, Proteobacteria, Actinobacteria, Bacteroidetes, Deinococcus-thermus, Gemmatimonadetes, Spirochaetes, Verrucomicrobia, Chloroflexi, Armatimonadetes or mixtures thereof.

13. A process as defined in claim 1, wherein the waste material depository contains soil.

14. A process as defined in claim 1, wherein the waste material depository comprises a landfill.

15. A process as defined in claim 1, wherein the encapsulated microorganism product contacts moisture that is already present in an environment in which the waste material depository resides.

16. A process as defined in claim 1, wherein the dehydrated polymer carrier comprises a crosslinked polyacrylate.

17. A process as defined in claim 1, wherein the dehydrated polymer carrier comprises a starch-based polymer, a cellulose-based polymer, agarose, or a crosslinked polyvinyl alcohol polymer.

18. A process as defined in claim 1, wherein the encapsulated microorganism product is in the form of flakes, chunks or cubes.

19. A process as defined in claim 1, wherein the encapsulated microorganism product comprises particles, fibers, or a granular material.

20. A process as defined in claim 1, wherein the at least one type of microorganism contained in the encapsulated microorganism product is selected based on environmental variables in which the waste material depository resides, the environmental variables comprising temperature, salinity, and concentration of oxygen.

21. A process as defined in claim 1, wherein at least 50%, such as at least 70%, such as at least 90% of the microorganisms contained in the encapsulated microorganism product are viable.

22. A process as defined in claim 4, wherein the bacterium is genetically modified by inserting a depolymerase gene into a chromosome of the microorganism by transduction or linear recombination.

23. A process as defined in claim 4, wherein the genetically modified microorganism is genetically modified by inserting a chromosomal vector into the microorganism that expresses the desired enzyme.

24. A process for decreasing polymer waste material comprising:
adding to a waste material depository a genetically modified microorganism, the waste material depository containing waste products comprising a polyhydroxyalkanoate polymer, the genetically modified microorganism having been modified to secrete an enzyme that degrades the polyhydroxyalkanoate polymer and increases a rate at which the polyhydroxyalkanoate polymer breaks down in the waste material depository.

25. A process as defined in claim 24, wherein the enzyme secreted by the microorganism comprises poly[R-3-hydroxybutyrate] depolymerase.

26. A process as defined in claim 24, wherein the at least one type of microorganism comprises at least one type of bacterium or archaean.

27. A process as defined in claim 24, wherein the waste products contained within the waste material depository comprise incontinence products, the incontinence products comprising a polyhydroxyalkanoate polymer.

28. A process as defined in claim 24, wherein the genetically modified microorganism comprises a bacterium selected from Firmicutes, Proteobacteria, Actinobacteria, Bacteroidetes, Deinococcus-thermus, Gemmatimonadetes, Spirochaetes, Verrucomicrobia, Chloroflexi, Armatimonadetes or mixtures thereof.

29. A process as defined in claim 24, wherein the waste material depository comprises a landfill.
